(19) United States
(12) Patent Application Publication
(10) Pub. No.: US 2012/0232120 A1
(43) Pub. Date: Sep. 13, 2012

(54) AMINO ACID AMIDES OF PHENOXYBUTYRIC ACID DERIVATIVES

(76) Inventors: IRAJ LALEZARI, LOUISVILLE, CO (US); JILL FABRICANT, CORONA DEL MAR, CA (US)

(21) Appl. No.: 13/415,650

(22) Filed: Mar. 8, 2012

Related U.S. Application Data
(60) Provisional application No. 61/464,679, filed on Mar. 8, 2011.

(51) Int. Cl. A61K 31/196; C07D 233/64; A61K 31/197; A61K 31/216; A61K 31/4172; C07C 233/75; C07C 233/47; A61K 8/43; A61K 8/42; A61K 8/49; A61P 25/28; A61P 29/00; A61P 25/00; A61P 9/10; A61P 15/00; A61P 3/10; A61P 27/02; A61Q 19/08; C07C 275/34

(52) U.S. Cl. 514/400; 562/439; 560/34; 548/338.1; 514/564; 514/563; 514/539; 562/455

(57) ABSTRACT

A compound of the formula:

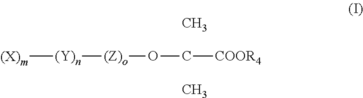

where X is phenyl substituted at the 3, 4 and 5 positions with R1, R2 or R3 which are selected from hydrogen, chloro, lower alkyl of 1 to 5 carbons, phenoxy, phenyl, naphthyl, or phenyl (lower) alkyl where the lower alkyl group has 1-5 carbon atoms and m is 0 or 1; Y is —CONH— or —NHCONH— where the nitrogen atoms are unsubstituted or substituted with other phenoxyisobutyric acid derivatives, or the residue of a phenoxyisobutyric acid and n is 0 or 1; Z is unsubstituted phenyl when m is 1 and n is 1; when Y is 0, X is 0; Z is also substituted.

### CROSS-REFERENCE TO RELATED APPLICATIONS

This application claims the priority of provisional application Ser. No. 61/464,679, filed Mar. 8, 2011

Not Applicable

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISK

Not Applicable

### REFERENCE TO A MICROFICHE APPENDIX

Not Applicable

### BACKGROUND OF THE INVENTION

1) Field of the Invention

This invention is directed to novel compounds which are useful in pharmaceutical compositions for human and veterinary use and in cosmetic compositions as well as in anti-aging compositions.

2) Description of Related Art

It is known in the art that elevated concentration of reducing sugars in the blood and in the intracellular environment results in the nonenzymatic formation of glycation and dehydration condensation complexes known as advanced glycation end-products or aminaglycation end products (AGEs). Nonenzymatic glycation is a complex series of reactions between reducing sugars and amino groups of proteins, lipids, and DNA. These complex products form on free amino groups on proteins, on lipids and on DNA (Bucala and Cerami, 1992; Bucala et al., 1993; Bucala et al., 1984). This phenomenon is called “browning” or a “Maillard” reaction and was discovered early in the last century by the food industry (Maillard, 1916). The reaction is initiated with the reversible formation of Schiffs base which undergoes rearrangement to form a stable Amadori product. Both Schiffs base and Amadori product further undergo a series of reactions through dicarbonyl intermediates to form AGEs. The significance of a similar process in biology became evident only after the discovery of the glycosylated hemoglobins and their increased presence in diabetic patients (Rahbar, 1968; Rahbar et al., 1969). In human diabetic patients and in animal models of diabetes, these nonenzymatic reactions are accelerated and cause increased AGE formation and increased glycation of long-lived proteins such as collagen, fibronectin, tubulin, lens crystallin, myelin, laminin and actin, in addition to hemoglobin and albumin, and also of LDL associated lipids and apoprotein. Moreover, brown pigments with spectral and fluorescent properties similar to those of late-stage Maillard products have also been found in vivo in association with several long-lived proteins such as crystalline lens proteins and collagen from aged individuals. An age-related linear increase in pigments was observed in human dura collagen between the ages of 20 to 90 years. AGE modified proteins increase slowly with aging and are thought to contribute to normal tissue remodeling. Their level increases markedly in diabetic patients as a result of sustained high blood sugar levels and lead to tissue damage through a variety of mechanisms including alteration of tissue protein structure and function, stimulation of cellular responses through AGE specific receptors or the generation of reactive oxygen species (ROS) (for a review see Boel et al., 1995). The structural and functional integrity of the affected molecules, which often have major roles in cellular functions, become disturbed by these modifications, with severe consequences on affected organs such as kidney, eye, nerve, and micro-vascular functions (Silbiger et al., 1993; Brownlee et al., 1985).

Structural changes on macromolecules by AGEs are known to accumulate under normal circumstances with increasing age. This accumulation is severely accelerated by diabetes and is strongly associated with hyperglycemia. For example, formation of AGE on protein in the subendothelial basement membrane causes extensive cross-link formation which leads to severe structural and functional changes in protein/protein and protein/cell interaction in the vascular wall (Haitoglou et al., 1992; Airaksinen et al., 1993).

Enhanced formation and accumulation of advanced glycation end products (AGEs) have been implicated as a major pathogenesis process leading to diabetic complications, normal aging, atherosclerosis and Alzheimer's disease. This process is accelerated by diabetes and has been postulated to contribute to the development of a range of diabetic complications including nephropathy (Nicholls and Mandel, 1989), retinopathy (Hammes et al., 1991) and neuropathy (Cameron et al., 1992). Particularly, tissue damage to the kidney by AGEs leads to progressive decline in renal function, end-stage renal disease (ESRD) (Makita et al., 1994), and accumulation of low-molecular-weight (LMW) AGE peptides (glycotoxins) (Koschinsky et al., 1997) in the serum of patients with ESRD (Makita et al., 1991). These low molecular weight (LMW)-AGEs can readily form new crosslinks with plasma or tissue components, e.g., low density lipoprotein (LDL) (Bucala et al., 1994) or collagen (Miyata et al., 1993) and accelerate the progression of tissue damage and morbidity in diabetics.

Direct evidence indicating the contribution of AGEs in the progression of diabetic complications in different lesions of the kidneys, the rat lens and in atherosclerosis has been reported (Vlassara et al., 1994; Vlassara et al., 1995; Horie et al., 1997; Matsumoto et al., 1997; Soulis-Liparota et al., 1991; Bucala and Vlassara, 1997; Bucala and Rahbar, 1998; Park et al., 1998). Indeed, the infusion of pre-formed AGEs into healthy rats induces glomerular hypertrophy and mesangial sclerosis, gene expression of matrix proteins and production of growth factors (Brownlee et al., 1991; Vlassara et al., 1995). Several lines of evidence indicate that the increase in reactive carbonyl intermediates (methylglyoxal, glycolaldehyde, glyoxal, 3-deoxyglucosone, malondialdehyde and hydroxynonenal) is the consequence of hyperglycemia in diabetes. “Carbonyl stress” leads to increased modification of proteins and lipids, followed by oxidant stress and tissue damage (Baynes and Thorpe, 1999; Onorato et al., 1998; McLellan et al., 1994). Further studies have revealed that aminoguanidine (AG), an inhibitor of AGE formation, ameliorates tissue impairment of glomeruli and reduces albuminuria in induced diabetic rats (Soulis-Liparota et al., 1991; Itakura et al., 1991). In humans, decreased levels of hemoglobin (Hb)-AGE (Makita et al., 1992) concomitant with amelioration of kidney function as the result of aminoguanidine therapy in diabetic patients, provides more evidence for the importance of AGEs in the pathogenesis of diabetic complications (Bucala and Vlassara, 1997).

The global prevalence of diabetes mellitus, in particular in the United States, afflicting millions of individuals with significant increases of morbidity and mortality, together with the great financial burden for the treatment of diabetic complications in this country, are major incentives to search for and develop drugs with a potential for preventing or treating complications of the disease. So far the mechanisms of hyperglycemia-induced tissue damage in diabetes are not well understood. However, four pathogenic mechanisms have been proposed, including increased polyol pathway activity, activation of specific protein kinase C (PKC) isoforms, formation and accumulation of advanced glycation endproducts, and increased generation of reactive oxygen species (ROS) (Kennedy and Lyons, 1997). Most recent immunohistochemical studies on different tissues from kidneys obtained from ESRD patients (Hone et al., 1997) and diabetic rat lenses (Matsumoto et al., 1997), by using specific antibodies against carboxymethyllysine (CML), pentosidine, the two known glycoxidation products and pyrraline, have localized these AGE components in different lesions of the kidneys and the rat lens, and have provided more evidence in favor of protein-AGE formation in close association with generation of ROS to be major factors in causing permanent and irreversible modification of tissue proteins. Therefore, inhibitors of AGE formation and antioxidants hold promise as effective means of prevention and treatment of diabetic complications.

The Diabetic Control and Complications Trial (DCCT), has identified hyperglycemia as the main risk factor for the development of diabetic complications (The Diabetes Control and Complications Trial Research Group, 1993). Compelling evidence identifies the formation of advanced glycation endproducts as the major pathogenic link between hyperglycemia and the long-term complications of diabetes (Makita et al., 1994; Koschinsky et al., 1997; Makita et al., 1993; Bucala et al., 1994; Bailey et al., 1998).

The reactions between reducing sugars and amino groups of proteins, lipids and DNA undergo a series of reactions through dicarbonyl intermediates to generate advanced glycation endproducts (Bucala and Cerami, 1992; Bucala et al., 1993; Bucala et al., 1984).

In human diabetic patients and in animal models of diabetes, AGE formation and accumulation of long-lived structural proteins and lipoproteins have been reported. Many reports indicate that glycation inactivates metabolic enzymes (Yan and Harding, 1999; Kato et al., 2000; Verbeke et al., 2000; O'Harte et al., 2000). The glycation-induced change of immunoglobin G is of particular interest. Reports of glycation of the Fab fragment of IgG in diabetic patients suggest that immune deficiency observed in these patients may be explained by this phenomenon (Lapolla et al., 2000). Furthermore, an association between IgM response to IgG damaged by glycation and disease activity in rheumatoid arthritis has been reported (Lucey et al., 2000). Also, impairment of high-density lipoprotein function by glycation has been described (Hedrick et al., 2000).

Methylglyoxal (MG) has recently received considerable attention as a common mediator and the most reactive dicarbonyl to form AGEs (Phillips and Thomalley, 1993; Beisswenger et al., 1998). It is also a source of reactive oxygen species (ROS) (free radicals) generation in the course of glycation reactions (Yim et al., 1995).

Nature has devised several humoral and cellular defense mechanisms to protect tissues from the deleterious effects of “carbonyl stress” and accumulation of AGEs, e.g., the glyoxylase systems (I and II) and aldose reductase catalyze the detoxification of MG to D-lactate (McLellan et al., 1994). Amadoriases are also a novel class of enzymes found in Aspergillus which catalyze the deglycation of Amadori products (Takahashi et al., 1997). Furthermore, several AGE-receptors have been characterized on the surface membranes of monocytes and on macrophage, endothelial, mesangial and hepatic cells. One of these receptors, RAGE, a member of the immunoglobulin superfamily, has been found to have a wide tissue distribution (Schmidt et al., 1994; Yan et al., 1997). The discovery of various natural defense mechanisms against glycation and AGE formation suggests an important role of AGEs in the pathogenesis of vascular and peripheral nerve damage in diabetes. MG binds to and irreversibly modifies arginine and lysine residues in proteins. MG modified proteins have been shown to be ligands for the AGE receptor (Westwood et al., 1997) indicating that MG modified proteins are analogous (Schalkwijk et al., 1998) to those found in AGEs. Furthermore, glycolaldehyde, a reactive intermediate in AGE formation, generates an active ligand for macrophage scavenger receptor (Nagai et al., 2000). The effects of MG on LDL have been characterized in vivo and in vitro (Bucala et al., 1993).

Lipid peroxidation of polyunsaturated fatty acids (PUFA), such as arachidonate, also yields carbonyl compounds; some are identical to those formed from carbohydrates (Al-Abed et al., 1996), such as MG and GO, and others are characteristic of lipids, such as malondialdehyde (MDA) and 4-hydroxynonenal (HNE) (Requena et al., 1997). The latter two carbonyl compounds produce lipoxidation products (Al-Abed et al., 1996; Requena et al., 1997). A recent report emphasizes the importance of lipid-derived MDA in the cross-linking of modified collagen and in diabetes mellitus (Slatter et al., 2000). A number of AGE compounds, both fluorophores and nonfluorescent, are involved in crosslinking proteins and have been characterized (Baynes and Thorpe, 1999). In addition to glucose derived AGE-protein crosslinks, AGE crosslinking also occurs between tissue proteins and AGE-containing peptide fragments formed from AGE-protein digestion and turnover. These reactive AGE-peptides, now called glycotoxins, are normally cleared by the kidneys. In diabetic patients, these glycotoxins react with the serum proteins and are a source for widespread tissue damage (He et al., 1999).

However, detailed information on the chemical nature of the crosslink structures remain unknown. The crosslinking structures characterized to date, on the basis of chemical and spectroscopic analyses, constitute only a small fraction of the AGE crosslinks which occur in vivo, with the major crosslinking structure(s) still unknown. Most recently, a novel acid-labile AGE-structure, N-omega-carboxymethylarginine (CMA), has been identified by enzymatic hydrolysis of collagen. Its concentration was found to be 100 times greater than the concentration of pentosidine (Iijima et al., 2000) and it is assumed to be a major AGE crosslinking structure.

In addition to aging and diabetes, the formation of AGEs has been linked with several other pathological conditions. IgM anti-IgG-AGE appears to be associated with clinical measurements of rheumatoid arthritis activity (Lucey et al., 2000). A correlation between AGEs and rheumatoid arthritis was also made in North American Indians (Newkirk et al., 1998). AGEs are present in brain plaques in Alzheimer's disease and the presence of AGEs may help promote the development of Alzheimer's disease (Durany et al., 1999; Munch et al., 1998; Munch et al., 1997). Uremic patients have elevated levels of serum AGEs compared to age-matched controls (Odani et al., 1999; Dawnay and Millar, 1998). AGEs have also been correlated with neurotoxicity (Kikuchi et al., 1999). AGE proteins have been associated with atherosclerosis in mice (Sano et al., 1999) and with atherosclerosis in persons undergoing hemodialysis (Takayama et al., 1998). A study in which aminoguanidine was fed to rabbits showed that increasing amounts of aminoguanidine led to reduced plaque formation in the aorta thus suggesting that advanced glycation may participate in atherogenesis and raising the possibility that inhibitors of advanced glycation may retard the process (Panagiotopoulos et al., 1998). Significant deposition of N(epsilon)-carboxymethyl lysine (CML), an advanced glycation endproduct, is seen in astrocytic hyaline inclusions in persons with familial amyotrophic lateral sclerosis but is not seen in normal control samples (Kato et al., 1999; Shibata et al., 1999). Cigarette smoking has also been linked to increased accumulation of AGEs on plasma low density lipoprotein, structural proteins in the vascular wall, and the lens proteins of the eye, with some of these effects possibly leading to pathogenesis of atherosclerosis and other diseases associated with tobacco usage (Nicholl and Bucala, 1998). Finally, a study in which aminoguanidine was fed to rats showed that the treatment protected against progressive cardiovascular and renal decline (Li et al., 1996). The mechanism of the inhibitory effects of aminoguanidine in the cascade of glycosylation events has been investigated. To date, the exact mechanism of AG-mediated inhibition of AGE formation is not completely known. Several lines of in vitro experiments resulted in contrasting conclusions. Briefly, elevated concentrations of reducing sugars cause reactions between carbohydrate carbonyl and protein amino groups leading to: 1. Reversible formation of Schiffs bases followed by 2. Amadori condensation/dehydration products such as 3-deoxyglucason (3-DG), a highly reactive dicarbonyl compound (Kato et al., 1990). 3. Irreversible and highly reactive advanced glycosylation endproducts. Examples of early Amadori products are ketoamines which undergo further condensation reactions to form late AGEs. A number of AGE products have been purified and characterized recently, each one constituting only minor fractions of the in vivo generated AGEs. Examples are pyrraline, pentosidine, carboxymethyl-lysine (CML), carboxyethyl-lysine (CEL), crossline, pyrrolopyridinium, methylglyoxal lysine dimer (MOLD), Arg-Lys imidazole, arginine pyridinium, cypentodine, piperidinedinone enol and alkyl, formyl, diglycosyl-pyrrole (Vlassara, 1994).

Analysis of glycation products formed in vitro on a synthetic peptide has demonstrated that aminoguanidine does not inhibit formation of early Amadori products (Edelstein and Brownlee, 1992). Similar conclusions were reached by analysis of glycation products formed on BSA (Requena et al., 1993). In both experiments AGE formation was strongly inhibited by AG as analyzed by fluorescence measurements and by mass spectral analysis. The mass spectral analysis did not detect peptide complexes with molecular mass corresponding to an incorporation of AG in the complex. Detailed mechanistic studies using NMR, mass spectroscopy and X-ray diffraction have shown that aminoguanidine reacts with AGE precursor 3-DG to form 3-amino-5- and 3-amino-6-substituted triazines (Hirsch et al., 992). In contrast, other experiments using labeled .sup.14C-AG with lens proteins suggest that AG becomes bound to the proteins and also reacts with the active aldose form of free sugars (Harding, 1990).

Several other potential drug candidates as AGE inhibitors have been reported. These studies evaluated the agent's ability to inhibit AGE formation and AGE-protein crosslinking compared to that of aminoguanidine (AG) through in vitro and in vivo evaluations (Nakamura et al., 1997; Kochakian et al., 1996). It is known that N-phenacylthiazolium bromide (PTB), which selectively cleaves AGE-derived protein cross-links in vitro and in vivo (Vasan et al., 1996; Ulrich and Zhang, 1997). The pharmacological ability to break irreversible AGE-mediated protein crosslinking offers potential therapeutic use.

It is well documented that early pharmaceutical intervention against the long-term consequences of hyperglycemia-induced crosslinking prevent the development of severe late complications of diabetes. The development of nontoxic and highly effective drugs that completely stop glucose-mediated crosslinking in the tissues and body fluids is a highly desirable goal. The prototype of the pharmaceutical compounds investigated both in vitro and in vivo to intervene with the formation of AGEs on proteins is aminoguanidine (AG), a small hydrazine-like compound (Brownlee et al., 1986). However, a number of other compounds were found to have such an inhibitory effect on AGE formation. Examples are D-lysine. (Sensi et al., 1993), desferrioxamine (Takagi et al., 1995), D-penicillamine (McPherson et al., 1988), thiamine pyrophosphate and pyridoxamine (Booth et al., 1997) which have no structural similarities to aminoguanidine.

A number of hydrazine-like and non-hydrazine compounds have been investigated. So far AG has been found to be the most useful inhibitor of glycation. AG is also a well known selective inhibitor of nitric oxide (NO) and can also have antioxidant effects (Tilton et al., 1993).

A number of other potential drug candidates to be used as AGE inhibitors have been discovered recently and evaluated both in vitro and in vivo (Nakamura et al., 1997; Soulis et al., 1997). While the success in studies with aminoguanidine and similar compounds is promising, the need to develop additional inhibitors of AGEs continues to exist in order to broaden the availability and the scope of this activity and therapeutic utility.

Amino acid compounds through their amino group can be bound to the carboxylic acid group of phenoxyisobutyric acid to form amide derivatives. These novel amides have increased activity as compared to the phenoxyisobutyric acid derivative from which they are derived. One reason for the increased activity is believed to be due to be that the amide derivatives have less affinity to serum albumin than the parent phenoxyisobutyric acid compounds and as a result have higher bioavailability. It is believed that the amides will also be more readily metabolized in the body.

The compounds of the invention may be synthesized by using general peptide synthesis methods including mixed anhydride and active esters. All amino acids including α, β, γ, ε etc. may be used to prepare the amides of the invention. The amino acids may be linear, cyclic or heterocyclic carboxylic acids. Alkali metal salts of the amides that are synthesized under aqueous conditions are water soluble and are preferred for administration of the compounds to a patient.

The present inventors have previously reported new classes of compounds which are aryl (and heterocyclic) ureido and aryl (and heterocyclic) carboxamido phenoxyisobutyric acids and also benzoic acid derivatives and related compounds as inhibitors of glycation and AGE formation (Rahbar et al., 1999; Rahbar et al., 2000; Rahbar et al., 2002). See also U.S. Pat. Nos. 5,093,367; 6,072,072; 6,337,350; 6,605,642 and 7,030,133 from which the structures of these patents are incorporated herein by reference as well as the methods of preparing the compounds disclosed in those patents. An elevated concentration of reducing sugars (i.e., glucose) in the blood and in the intracellular environment of an animal, namely a human, typically results in the nonenzymatic formation of glycation and dehydration condensation complexes known as advanced glycation end-products (AGE). These AGE complex products form on free amino groups, on proteins, on lipids and on DNA (Bucala and Cerami, Adv Pharmacol 23:1-34, 1992; Bucala et al., Proc Natl Acad Sci 90:6434-6438, 1993; Bucala et al Proc Natl. Acad Sci 81:105-109, 1984). This phenomenon is called “browning” or a “Maillard” reaction and was discovered early in the last century by the food industry (Maillard, Ann Chim 5:258-317, 1916). The significance of a similar process in biology became evident only after the discovery of the glycosylated hemoglobins and their increased presence in diabetic patients (Rahbar, Clin Chim Acta 20:381-5, 1968; Rahbar et al., Biochem Biophys Res Commun 36:838-43, 1969). A diabetic patient's AGE level increases markedly as a result of sustained high blood sugar levels and often leads to tissue damage through a variety of mechanisms including alteration of tissue protein structure and function, stimulation of cellular responses through AGE specific receptors and/or the generation of reactive oxygen species (ROS) (for a recent review see Boel et al., J Diabetes Complications 9:104-29, 1995). These AGE have been shown to cause complications in patients suffering from various pathological conditions, including, but not limited to, diabetes mellitus, rheumatoid arthritis, Alzheimer's Disease, uremia and in atherosclerosis in persons undergoing hemodialysis.

### SUMMARY OF THE INVENTION

The invention is directed to compounds of formula I, as well as pharmaceutical and compositions thereof for cosmetic and antiaging use and methods of using said compounds:

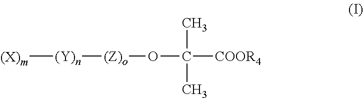

where X is phenyl substituted at the 3, 4 and 5 positions with R1, R2 or R3 which are selected from hydrogen, chloro, lower alkyl of 1 to 5 carbons, phenoxy, phenyl, naphthyl, or phenyl (lower) alkyl where the lower alkyl group has 1-5 carbon atoms and m is 0 or 1; Y is —CONH— or —NHCONH— where the nitrogen atoms are unsubstituted or substituted with other phenoxyisobutyric acid derivatives, or the residue of a phenoxyisobutyric acid and n is 0 or 1; Z is unsubstituted phenyl when m is 1 and n is 1; when Y is 0, X is 0; Z is phenyl substituted at the 3, 4 and 5 positions with R1, R2 or R3 which are selected from hydrogen, chloro, lower alkyl of 1 to 5 carbons, phenoxy, phenyl, naphthyl, or phenyl (lower) alkyl where the lower alkyl group has 1-5 carbon atoms; and R4 is hydrogen alkyl of 1-5 carbon atoms; aminoalkyl COORS, where the alkyl group may be substituted with a carboxyl or histidine substituted lower alkyl group having 1-5 carbons and, or is a cycloalkyl group; R5 is hydrogen or lower alkyl of 1-5 carbons or an acyl-lower alkyl group where the lower alkyl group has 1-5 carbons

Examples of suitable amino acid containing compounds include amino acid compounds such as valine, 4-aminoisobutyric acid, leucine, 3-amino isobutyric acid, isoleucine, asparagine, methionine, phenylalanine, proline, tryptophan, glutamic acid, cysteine, glutamine, arginine, histidine, lysine, aspartic acid, glycione, serine, threonine, tyrosine, ε-aminocaproic acid, 1-aminocyclohexanecarboxylic acid, cystine, arginine and the like. Other amino acids include taurine and all unnatural synthetic amino acids.

The compounds of formula 1 may be used topically or systemically. The topical uses include anti-aging effects that are achieved by mixing the compounds with a cosmetic base that is applied to the skin using an amount that forms a thin film. The systemic uses include reduction of serum cholesterol, anti-glycation in diabetes, treatment of atherosclerosis, neuro-degenerative diseases, anti-aging of the skin by the modification of collagen; and prevention of skin wrinkles.

### DETAILED DESCRIPTION OF THE INVENTION

The phenoxyisobutyric amides of the invention may be made by reacting a mixed anhydride of the phenoxyisobutyric acid derivative in a conventional peptide synthesis using a non-aqueous solvent such as tetrahydrofuran with equimolar quantities of triethyl amine and ethyl chloroformate using an ice bath with stirring. The reaction product is added to a solution of one mole of an amino acid (or 0.5 mole of a diamino acid such as cysteine or lysine dissolved in a 2 molar aqueous solution of NaOH (cooled to ice bath temperature). After 0.5 hours of stirring at room temperature, the mixture is warmed to 60° C. while stirring. At this time the tetrahydrofuran is evaporated and the residue is diluted with water and acidified with an acid such as 0.1N HCl or 0.1N citric acid. The solution is cooled to crystallize the product.

Alternatively, 4-aminophenoxybutyric acid may be converted to its methyl ester using an ice cold salt solution (temperature of 0° C.) of Normal thionyl chloride in methanol. The methyl ester hydrochloride salt may be reacted with any appropriate aryl isocyanate to obtain the methyl ester of aryliminocarbonyl phenoxyisobutyric acid methyl ester. Using a hot (temperature ˜50° C.) Normal NaOH solution, the methyl group of the ester group is removed. The free acid reacts with the methyl ester of any amino acid in ethyl acetate solution in the presence of dicylcohexylcarbodiimide to yield the methyl ester of the of the amino acid derivative. The methyl ester may be removed by boiling NaOH followed by acidification.

An additional procedure for preparing compounds according to the invention is the formation of active esters. The most successful ester is N-hydroxy succinimide of clofibric acid derivatives which are prepared using the dried acid dissolved in tetrahydrofuran or dioxane with equimolar quantities of N-hydroxy succinimide and one mole of dicyclohexylcarbodiimide at room temperature with stirring overnight. After the addition of a small quantity of water to decompose the dicyclohexylcarbodiimide, the reaction mixture is filtered and eventually extracted with ethyl acetate to free the product from any dicyclohexylurea and obtain the pure product in high yields.

The product prepared by this method is identical to the product prepared by using the mixed anhydride method or by the use of esters of amino acids as described above.

A further method of preparing the amino acid amides of the invention is by dissolving the methyl or ethyl esters of the amino acid in ethyl; acetate and reacting the dissolved ester with the appropriate phenoxybutyric acid derivative in the presence of dicyclohexylcarbodiimide. After the reaction is complete, the dicyclohexyldicarbodiimide is decomposed by the addition of a small quantity of water. The solvent is removed by evaporation and the free amide is precipitated by the addition of an appropriate acid.

The compounds of the present invention inhibit the nonenzymatic formation of glycation and dehydration condensation complexes known as advanced glycation end-products (AGE). In one embodiment of the present invention, a method is provided for administering a medication that inhibits the nonenzymatic formation of glycation and dehydration condensation complexes known as advanced glycation end-products (AGE) to a subject in need thereof, comprising providing at least one medication that inhibits the nonenzymatic formation of AGE complexes; and administering the medication to an patient wherein the nonenzymatic formation of AGE complexes is inhibited.

In another embodiment of the method, the administering step comprises a route of administration selected from the group consisting of oral, sublingual, intravenous, intracardiac, intraspinal, intraosseous, intraarticular, intrasynovial, intracutaneous, subcutaneous, intramuscular, epicutaneous, transdermal, conjunctival, intraocular, intranasal, aural, intrarespiratory, rectal, vaginal and urethral. In another embodiment, the administering step comprises providing the medication on an implantable medical device.

While these medications are typically parameter specific medications, they are efficacious in wound healing, in scar reduction and in the treatment of burns including damage caused by laser cosmetic therapy. For example, a compound that inhibits the formation of AGE complexes may be directly applied to in a conventional hydrophilic or oleophilic ointment base, or incorporated within, a medical device (i.e., a wound dressing, patch, etc.) and applied to a patient's skin to aid the would healing process.

Any method of administering the medication(s) discussed herein is contemplated. While it is understood by one skilled in the art that the method of administration may depend on patient specific factors, the methods of administration include, but are not limited to, generally parenteral and non-parenteral administration. More specifically, the routes of administration include, but are not limited to oral, sublingual, intravenous, intracardiac, intraspinal, intraosseous, intraarticular, intrasynovial, intracutaneous, subcutaneous, intramuscular, epicutaneous, transdermal, conjunctival, intraocular, intranasal, aural, intrarespiratory, rectal, vaginal, urethral, etc. Typically, an oral route of administration is preferred.

Of course, it is understood that the medication will be administered in the appropriate pharmaceutical dosage, depending on the route of administration. For example, an oral dosage form may be administered in at least one of the following pharmaceutical dosage forms: tablet; capsule; solution; syrup; elixir; suspension; magma; gel; and/or powder. A sublingual preparation may be administered in at least one of the following pharmaceutical dosage forms: tablet; troche; and/or lozenge. A parenteral dosage form may be administered in at least one of the following pharmaceutical dosage forms: solution and/or suspension. An epicutaneous/transdermal dosage form may be administered in at least one of the following pharmaceutical dosage forms: ointment; cream; infusion pump; paste; plaster; powder; aerosol; lotion; transdermal patch/disc/solution. A conjunctival dosage form may be administered in at least one of the following pharmaceutical dosage forms: contact lens insert and/or ointment. An intraocular/intraaural dosage form may be administered in at least one of the following pharmaceutical dosage forms: solution and/or suspension. An intranasal dosage form may be administered in at least one of the following pharmaceutical dosage forms: solution; spray; inhalant and/or ointment. An intrarespiratory dosage form may be administered in at least one of the following pharmaceutical dosage forms: aerosol and/or powder. A rectal dosage form may be administered in at least one of the following pharmaceutical dosage forms: solution; ointment and/or suppository. A vaginal dosage form may be administered in at least one of the following pharmaceutical dosage forms: solution; ointment; emulsion foam; tablet; insert/suppository/sponge. A urethral dosage form may be administered in at least one of the following pharmaceutical dosage forms: solution and/or suppository.

The above-noted dosage form(s) may include at least one medication disclosed herein, either alone or in combination with at least one other medication disclosed herein or with a medication not disclosed herein and/or in combination with at least one inert pharmaceutical excipient. These medications may have any release profile including, but not limited to, an immediate

release, a controlled release and/or a delayed release profile.

The medical devices include, but are not limited to, implantable medical devices such as, but not limited to, stents (both vascular and urethral), deposition implants (implantable medication releasing device), and/or a medication delivery pumps. Also, contemplated herein are topically applied medical devices including, but not limited to, patches, gauze, wraps, appliques, dressings, coverings, etc. In the case of a medical device, at least one medication may be releasably applied either to at least a portion of the surface of the device, or to a material applied to the surface of a device. Alternatively, at least one medication may be absorbed and/or adsorbed into or onto the device material so long as the medication may be released from the material at a later time.

The medication may be releaseably applied to the medical device via any industrially acceptable method, including, but not limited to, spray coating, a waterfall method, heat annealing, etc., however, spray coating is typically preferred. Alternatively, the medical device may include at least one medication, wherein the medication is absorbed and/or adsorbed into or onto the medical device. This may be done by any industrially acceptable method. Also, it is contemplated herein that a medical device may include both at least one medication releasably applied to the medical device itself and/or a coating applied to the device and at least one medication absorbed and/or adsorbed into or onto the medical device itself.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

In the course of screening different classes of organic compounds for investigation of their possible inhibitory and/or breaking effects on advanced glycation endproducts (AGEs), it has been found that most of the phenylureido substituted phenoxy propionic acid derivatives tested have inhibitory effects and several of these compounds were potent inhibitors of AGE-formation at concentrations much lower than an equally inhibiting concentration of aminoguanidine.

The mechanism by which this class of compounds inhibits glycation and/or breaking of glycation, AGE-formation, and crosslinking is yet to be known in full. Three major mechanisms include anti-inflammatory (PPAR RAGE), transient-metal-chelation such as copper and iron, and free-radical scavenging or trapping of reactive carbonyl intermediates have been proposed to be responsible for AGE-inhibitory function of known AGE-inhibitors.

The mechanism of the inhibitory activities of guanidino compound inhibitors such as two known inhibitors of glycation (aminoguanidine and metformin) is that they are postulated to trap MG and other alpha.-dicarbonyl intermediates of glycation. A most recent study has documented the reaction of metformin with MG and glyoxal (GO), forming guanidino-dicarbonyl adducts further supporting this idea (Ruggiero-Lopez et al., 1999).

Using known assay methods specific for the early (Amadori) and late (post-Amadori) stages of glycation has revealed that some inhibitors have greater effects in the early stages and some in the late stages of glycation. However, most of the inhibitor compounds we have investigated are multistage inhibitors. The reaction of reducing sugars with .alpha.- and .epsilon.-amino groups of proteins is not a random process but rather a site specific reaction which depends on the nature and the vicinity of these chemical groups. The future task is to specifically define the site and/or sites of interaction of an inhibitor compound in the complex series of reactions and intermediate substrates, leading to AGE formation and cross-linking.

The development of the novel inhibitors of glycation, AGE formation, and AGE-protein crosslinking expands the existing arsenals of inhibitors of glycation reaction that can find therapeutic applications for the prevention of diabetic complications, as well as the prevention of other diseases associated with increased glycation of proteins or lipids. Furthermore, the availability of these compounds may prove useful as tools to study the cascade of reactions and intermediate substrate in the process of AGE-formation and AGE-protein cross-linking.

The compounds of the invention and their useful compositions utilized in the present invention contain agents capable of reacting with the highly active carbonyl intermediate of an early glycation product thereby preventing those early products from later forming the advanced glycation end products or in the alternative as agents for “breaking” or reversing the AGE complexes after they form protein crosslinked compounds which cause protein aging. Doses of 1-1000 mg per day may be used to inhibit the formation of AGE complexes or to break AGE complexes depending on the desired effect and the observed response in a patient. The formation of AGE has been linked to several pathologies which may be treated according to the invention including chronic inflammation such as dry eyes, neuropathy, atherosclerosis, retinopathy, Alzheimer's disease, erectile dysfunction and diabetes. The compounds of the invention are useful for the treatment of pre-diabetes, Type I and Type II diabetes as well as the prevention and/or treatment of diabetic complications such as elevated cholesterol, retinopathy, kidney damage, circulatory disorders, neuropathy and the like. The compounds of the invention also have activity against rheumatoid arthritis, Alzheimer's disease, Wilson's disease, atherosclerosis, neurodegenerative diseases, such as multiple sclerosis, neurotoxins and metabolic syndrome. An oral dose for these conditions is preferred but other routes of administration may be utilized. An effective amount of an oral dose will be from 1-1000 mg daily preferable given in divided doses. It is presently contemplated that a dose of 250-500 mg daily would be preferred.

Other utilities envisioned for the present invention are prevention of aging of the skin by exerting an anti-aging effect that reduces wrinkles and makes the skin smoother. The compounds also inhibit spoilage of proteins in foodstuffs such as the browning reaction seen in certain fruits. The present agents are also useful in the area of oral hygiene as they prevent discoloration of teeth and may be used as solutions or dispersions in water or a cream at a concentration of 0.1 to 10% by weight. and used as a cosmetic on the skin to improve the smoothness, texture, appearance and to prevent or treat aging of the skin. A particular use is the application of compounds to skin for the purpose of increasing the collagen content which will inhibit or reverse environmental aging effects. The compounds of the invention reduce the amount of MMP9 in the skin which makes the compounds useful for traumatic and surgical wounds. They may be used systemically or topically for scleroderma, acne, psoriasis, inflammation, antioxidant effects or for chelation of metals. For topical use, the compounds may be added to hydrophilic or oleophilic cosmetic bases in amounts of 0.01 to 10% by weight, and preferably 1-5% or they may be applied as a solution, dispersion or gel. The cosmetic bases include any commercially available cosmetic cream that is designed for application to the skin. For systemic use, the compounds may be administered orally at a dose of 1-1000 mg daily in divided doses. The dose will be adjusted depending on the observed effects using conventional dosing techniques. The compounds of the invention have PPAR activity which is an acronym for peroxisome proliferator activated receptor which are a group of receptor isoforms which exist across biology. They are intimately connected to cellular metabolism (carbohydrate, lipid and protein) and cell differentiation. They are also transcript factors. Several types of PPARs have been identified: alpha, gamma 1, 2 and 3 as well as delta or beta. The alpha form is expressed in liver, kidney, heart, adipose tissues as well as in other tissues. The gamma 1 form is expressed in virtually all tissues including heart, muscle, colon, kidney, pancreas and spleen tissues. The gamma 2 form is expressed mainly in adipose tissue (30 amino acids or longer while gamma 3 is expressed in macrophage, large intestine and white adipose tissue. Delta is expressed in many tissues but mainly in brain, adipose tissue and skin. PPARs dimerize with the retinoid receptor and bind to specific regions on the DNA of the largest genes and when PPAR binds to its ligand, transcription of target genes is increased or decreased depending on the gene. The PPAR activity of the compounds of the invention is a property that confirms that the compounds of the invention are useful as antidiabetic compounds in the manner that the PPAR active compound pioglitazone is useful when administered orally to diabetics. The dose may be from 1 to 1000 mg orally and preferably 250-500 mg orally, daily basis given in divided doses.

To aid in the administration, the compound may be combined with a pharmaceutical acceptable diluent or carrier to form a pharmaceutical dosage form. The dosage form can be a liquid, solid, gel for immediate release or controlled release. Common pharmaceutical diluents or carriers are described in the Handbook of Pharmaceutical Excipients, 4^{th} addition, the United States Pharmacopeia, and Remington's Pharmaceutical Science. The compounds of the invention may be prepared using one of the following methods: mixed anhydride method (A); active ester method (B) or the amino acid ester method (C).

The reaction scheme for Method (A) is as follows:

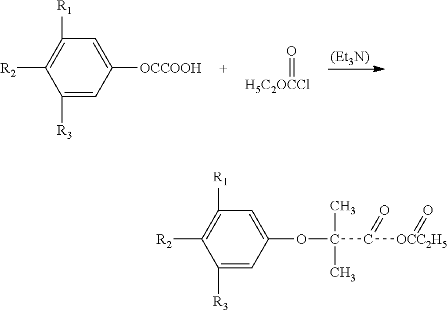

where R1, R2, R3 and R4 are as above described.

The reaction scheme for Method (B) is as follows:

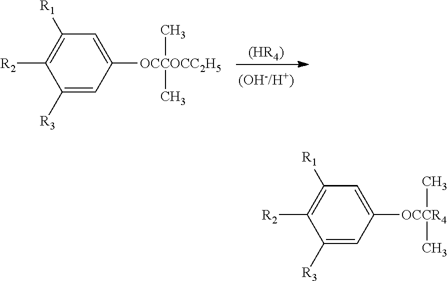

where R1, R2, R3 and R4 are as above described; R5 is alkyl of 1-10 carbon atoms or phenyl or naphthyl.

The reaction scheme for Method (C) is as follows:

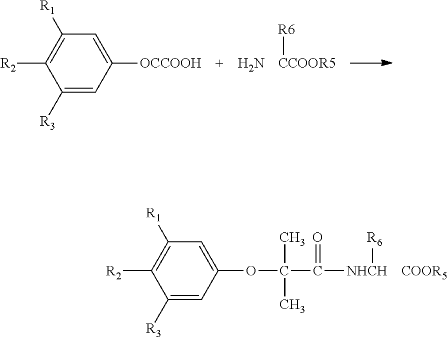

where R1, R2, R3, R4 and R5 are as above described and R6 is an alkyl of 1-10 carbon atoms, phenyl, naphthyl, alkylphenyl, phenylalkyl or alkylphenylalkyl where the alkyl group is from 1-10 carbon atoms.

### Example 1

To a stirring mixture of 1 mole of clofibric acid (2.145 g) in 25 ml of tetrahydrofuran and 1.4 ml of triethyl amine on an ice-salt bath is slowly added 1 ml of ethyl chloroformate and a solution of 1.3 g (0.01 mole) of 4-aminobutyric acid in 7.5 ml of 2N aqueous sodium hydroxide with 15 ml of tetrahydrofuran. After one hour of stirring at room temperature, most of the tetrahydrofuran evaporated. The aqueous solution was filtered over Celite and acidified with citric acid. After two hours in a refrigerator, the solid was filtered and air dried giving 2.7 g of product (approximately 90% yield) mp 95-97° C. The white powder may be crystallized from aqueous isopropanol without changing the melting point.

This compound has the following formula:

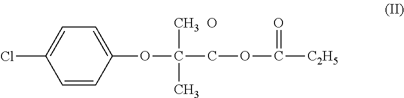

### Example 2

One mole of the following compound:

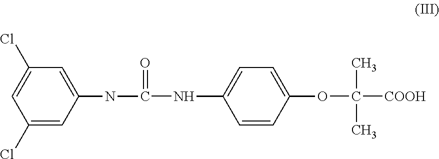

is dissolved in 8 ml of tetrahydrofuran, 0.15 ml of triethyl amine and 0.1 ml (approximately 0.1 mmole) of ethylchloroformate is added to and cooled on an ice salt bath for 0.5 hours. A solution of 3-aminobutyric acid in 1.2 ml of aqueous 1 N NaOH is added to the previously prepared solution and an off white precipitate is formed. After the addition of 15 ml of water and acidification with citric acid, the precipitate was filtered, washed with water and dried giving 456 mg (97% yield) of a white product mp 96-98° C. having the following formula:

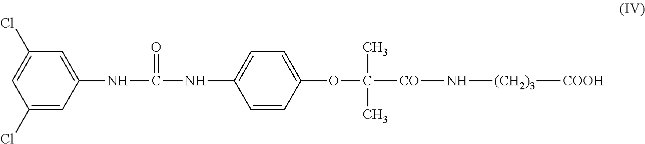

### Example 3

To a cold solution of 382 mg of a compound of the formula:

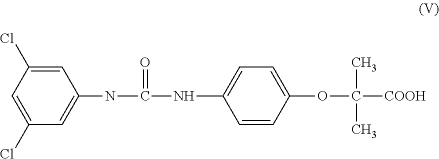

in 8 ml of tetrahydrofuran is added 0.15 ml (approximately 1 mmole) of triethylamine and 0.1 ml (approximately 1 mmole) of ethylchloroformate and the mixture is stirred for 0.5 hours and a solution of 107 mg (1.1 mmole) of glycine in 2 ml of aqueous 1 N NaOH with 3 ml of water is added and stirred for 0.5 hours at room temperature prior to adding 20 ml of water. The tetrahydrofuran is evaporated, the mixture is cooled and acidified with citric acid. A off white precipitate forms which is washed with water and dried to give 400 mg (yield 90%) mp 101-103° C. of a product of the formula:

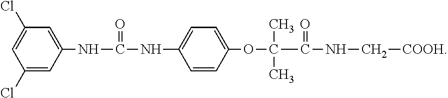

### Example 4

2.5 mmoles of a compound of the formula:

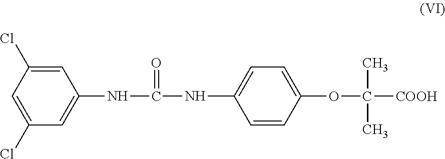

is added in 15 ml of tetrahydrofuran containing 0.25 ml of triethylamine, 483 mg of the methyl ester of 1-amino cyclohexane-1-carboxylic acid hydrochloride and 0.515 g (2.5 mmole) of dicyclohexylcarbodiimide. The resulting mixture is stirred at room temperature for an additional 6 hours. The mixture is then suction filtered and the solid precipitate is washed with 8 ml of tetrahydrofuran. Evaporation of the tetrahydrofuran gives a light colored oil that is dissolved in carbon tetrachloride. The NMR shows that some unreacted material is present The oil is dissolved in 15 ml of isopropanol and 5 ml of aqueous 2N NaOH solution, warmed to and stirred for 1 hour. The resulting mixture is acidified with 0.1N hydrochloric acid and cooled to 0° C. to give 700 mg (54.3% yield) of a product having the formula:

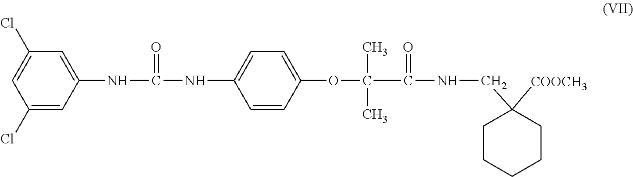

### Example 5

To a cold solution (0° C.) of 383 mg (1 mmole) 3,5-dichlorophenylureidophenoxyisobutyric acid in 8 ml of tetrahydrofuran, 0.15 ml of triethylamine and 0.1 ml of ethylchloroformate are added. After 0.5 hours of stirring, a solution of 177 mg (1.1 mmole) of the γ-methyl ester of glutamic acid in 1 ml of aqueous 1N NaOH solution with 5 ml of water and 5 ml of tetrahydrofuran is added with stirring for 1 hour at room temperature. The tetrahydrofuran was evaporated until a gum is obtained. The gum was acidified with 0.1N hydrochloric acid and extracted with ethyl acetate. The resulting product is then dissolved in 2N sodium carbonate solution, filtered, acidified with 0.1 N HCl, cooled in a refrigerator to give 316 mg of a crystalline compound (mp 97-99° (approximate yield 60%) having the formula:

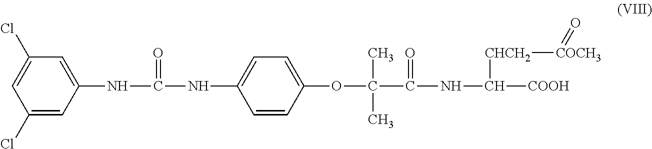

### Example 6

4-chlorobenzoyl chloride (1 mole) is added drop wise to a pyridine solution of 4-aminophenoxyisobutyric acid (1 mole) while being cooled on a water bath at 0° C. After the mixture is stirred for 1 hour, the pyridine is decomposed with) 1N HCl and a white crystalline product is obtained in the theoretical yield with a mp of 179-180° C. The product is of the following formula:

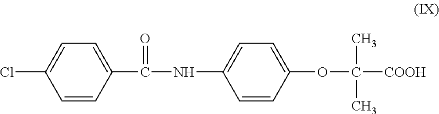

651 mg (2 mmoles) of a compound of Formula (IX) in 10 ml of tetrahydrofuran is combined with 0.2 ml of ethylchloroformate and 0.3 ml of triethyl amine with stirring on an ice salt bath for one hour. To that mixture, is added a cold (0° C.) solution of 240 mg (1 mmole) of cystine in 2 ml of aqueous 1N NaOH with 3 ml of water in 3 ml of tetrahydrofuran followed by an additional 3 ml of aqueous NaOH at room temperature. The resulting mixture is stirred for one hour and the clear solution acidified with 0.1N HCL and refrigerated for 24 hours prior to filtering off 820 mg (approximate yield 94%) of the compound of formula (X) mp (softening) 115-117° C. and melting at 125°. The product is recrystallized by dissolving the compound in chloroform, cooling the mixture to 0° C. followed by the addition of excess petroleum ether which crystallizes as a white powder without a change in the melting point.

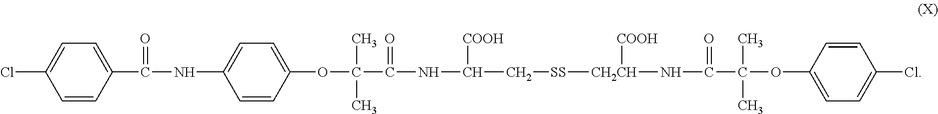

### Example 7

To a solution of 2.145 g (0.01 mole) of clofibric acid in 20 ml of tetrahydrofuran, cooled on an ice salt bath, is added 1.4 ml of triethyl amine and 1 ml of ethylchloroformate. After stirring the mixture for 0.5 hours, a solution of 1.312 g (0.1 mole) of ε-amino caproic acid in 9 ml of 1N aqueous NaOH is added and stirred for one hour at room temperature. Most of the tetrahydrofuran is evaporated and the mixture is acidified with 0.1N HCl to give an oil which is extracted with ethyl acetate. After evaporation of the ethyl acetate 3.235 g approximate yield 98.5%) of a colorless oil of formula (XI). The structure of formula (XI) is confirmed with NMR spectroscopy in carbon tetrachloride. Thin layer chromatography on silica gel showed a single spot.

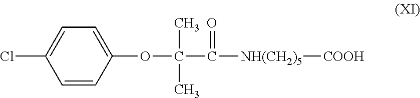

### Example 8

To a solution of 3.85 g (0.01 mole) of 4,5-dichlorophenylureido phenoxyisobutyric acid in 3.0 ml of tetrahydrofuran cooled on an ice salt bath while stirring, is added, dropwise, 1.5 ml of ethylchloroformate to form a mixed anhydride solution and a solution of L-histidine (1.2 g in 0.5 g of LiOH and 10 ml of water) is added all at once to the mixed anhydride solution with evolution of carbon dioxide. The mixture is stirred overnight and the next day is warmed to a temperature of 50° C. and 30 ml of water is added while an air flow is used to remove the tetrahydrofuran. Atr this time is added sufficient HCl to yield a gum to obtain a white residue of formula (XII) mp (soft) 175°; decomposition mp 214° C. with an approximate theoretical yield. The NMR was consistent with the structure of L-4(3,5-dichlorophenoxyureidophenoxy)isobutyryl histidine hydrochloride.

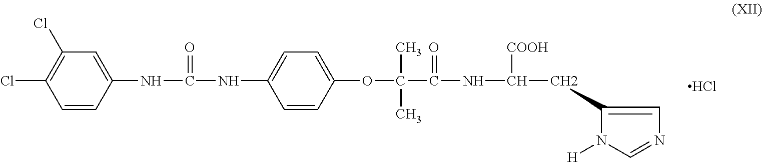

1. A compound of the formula: 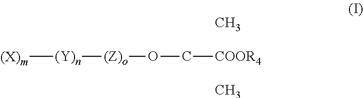
where X is phenyl substituted at the 3, 4 and 5 positions with R1, R2 or R3 which are selected from hydrogen, chloro, lower alkyl of 1 to 5 carbons, phenoxy, phenyl, naphthyl, or phenyl (lower) alkyl where the lower alkyl group has 1-5 carbon atoms and m is 0 or 1; Y is —CONH— or —NHCONH— where the nitrogen atoms are unsubstituted or substituted with other phenoxyisobutyric acid derivatives, or the residue of a phenoxyisobutyric acid and n is 0 or 1; Z is unsubstituted phenyl when m is 1 and n is 1; when Y is 0, X is 0; Z is phenyl substituted at the 3, 4 and 5 positions with R1, R2 or R3 which are selected from hydrogen, chloro, lower alkyl of 1 to 5 carbons, phenoxy, phenyl, naphthyl, or phenyl (lower) alkyl where the lower alkyl group has 1-5 carbon atoms; and R4 is hydrogen; alkyl of 1-5 carbon atoms; aminoalkyl COOR5, where the alkyl group may be substituted with a carboxyl or histidine substituted lower alkyl group having 1-5 carbons and, or is a cycloalkyl group; R5 is hydrogen or lower alkyl of 1-5 carbons or an acyl-lower alkyl group where the lower alkyl group has 1-5 carbons.

2. (canceled)

3. A compound as defined in claim 1 wherein R₁ and R₃ are hydrogen, R₂ is chloro and the amino acid is 4-aminobutyric acid.

4. A compound as defined in claim 1 wherein R₁ and R₃ are chloro, R₂ is hydrogen and the amino acid is 3-aminobutyric acid.

5. A compound as defined in claim 1 wherein R₁ and R₃ are hydrogen, R₂ is chloro and the amino acid is glycine.

6. A compound as defined in claim 1 wherein R₁ and R₃ are chloro, R₂ is hydrogen and the amino acid is 1-aminocyclohexanecarboxylic acid.

7. A compound as defined in claim 1 wherein R₁ and R₃ are hydrogen, R₂ is chloro and the amino acid is cystine.

8. A compound as defined in claim 1 wherein R₁ and R₃ are chloro, R₂ is hydrogen and the amino acid is ε-aminocaproic acid.

9. A compound as defined in claim 1 wherein R₁ and R₃ are hydrogen, R₃ is chloro and the amino acid is histidine.

10. A pharmaceutical composition comprising a pharmaceutical carrier and a compound as defined in claim 1.

11. A pharmaceutical composition as defined in claim 10 wherein the compound is derived from a compound of formula (I) and the amino acid is selected from the group consisting of valine, leucine, isoleucine, asparagine, methionine, phenylalanine, proline, tryptophan, glutamic acid, cysteine, glutamine, arginine, histidine, lysine, aspartic acid, glycione, serine, threonine, tyrosine, ε-aminocaproic acid, 1-aminocyclohexanecarboxylic acid, cystine and arginine.

12. A pharmaceutical composition as defined in claim 10 wherein the compound is a compound of formula (I) where R₁ and R3 are hydrogen, R₂ is chloro and the amino acid is 4-aminobutyric acid.

13. A pharmaceutical composition as defined in claim 10 wherein the compound is a compound of formula 1 wherein R₁ and R3 are chloro, R₂ is hydrogen and the amino acid is 3-aminobutyric acid.

14. A pharmaceutical composition as defined in claim 10 wherein the compound is a compound of formula (I) wherein R₁ and R3 are chloro, R₂ is hydrogen and the amino acid is glycine.

15. A pharmaceutical composition as defined in claim 10 wherein the compound is a compound of formula (I) wherein R1 and R₃ are chloro, R₂ is hydrogen and the amino acid is 1-aminocyclohexanecarboxylic acid.

16. A pharmaceutical composition as defined in claim 10 wherein the compound is a compound of formula (I) wherein R₁ and R₃ are hydrogen, R₂ is chloro and the amino acid is cystine.

17. A pharmaceutical composition as defined in claim 10 wherein the compound is a compound of formula (I) wherein R₁ and R₃ are chloro, R₂ is hydrogen and the amino acid is ε-aminocaproic acid.

18. A method of preventing the formation of advanced glycation end products or breaking advanced glycation end products which comprises administering an amount of a compound of formula (I), to a patient who may form or has formed advanced glycation end products, which is effective to prevent or break said glycation end products.

19. A method of treating a condition selected from the group consisting of chronic inflammation, neuropathy, atherosclerosis, retinopathy, Alzheimer's disease, erectile dysfunction and diabetes which comprises administering to a patient having a condition selected from the group consisting of chronic inflammation, neuropathy, atherosclerosis, retinopathy, Alzheimer's disease, erectile dysfunction, pre-diabetes and diabetes, an amount of a compound selected from formula (I) which is effective to treat said condition.

20. A cosmetic composition comprising a cosmetic carrier and a compound as defined in claim 1.

21-29. (canceled)
